(19) United States
(12) Patent Application Publication
(10) Pub. No.: US 2004/0235724 A1
(43) Pub. Date: Nov. 25, 2004

(54) Use of timp-1 as an immunosuppressive

(76) Inventors: Wolfgang E. Berdel, Berlin (DE); Elisabeth Oelmann, Munster (DE)

Correspondence Address: ARNOLD & PORTER LLP, ATTN: IP DOCKETING DEPT., 555 TWELFTH STREET, N.W., WASHINGTON, DC, 20004-1206, US

(21) Appl. No.: 10/486,090

(22) PCT Filed: Aug. 5, 2002
(86) PCT No.: PCT/EP02/08733

(30) Foreign Application Priority Data
Aug. 6, 2001 (DE) 101 38 550.1

(51) Int. Cl. A61K 38/08; A61K 38/10; A61K 38/17

(52) U.S. Cl. 514/12; 514/13; 514/14; 514/15; 514/16; 514/17

(57) ABSTRACT

The present invention relates to the use of specific inhibitors of metalloproteinases (the so-called “tissue inhibitor of metalloproteinases 1”; hereinafter “TIMP-1”) for the production of a pharmaceutical composition for the treatment of diseases or disorders characterised by an increased immunological activity.

### CROSS-REFERENCE TO RELATED APPLICATIONS

This application is a national phase application under 35 U.S.C. § 371 of International Application Number PCT/EP02/08733, filed Aug. 5, 2002, the disclosure of which is hereby incorporated by reference in its entirety, and claims the benefit of German Patent Application Number 101 38 550.1, filed Aug. 6, 2001.

### INCORPORATION OF SEQUENCE LISTING

A paper copy of the Sequence Listing and a computer readable form of the sequence listing on diskette, containing the file named “19235.002.seqlist.txt”, which is 6,384 bytes in size (measured in MS-DOS), and which was recorded on Feb. 6, 2004, are herein incorporated by reference.

### BACKGROUND OF THE INVENTION

The present invention relates to the use of specific inhibitors of metalloproteinases (the so-called “tissue inhibitor of metalloproteinases 1”; hereinafter “TIMP-1”) for the production of a pharmaceutical composition for the treatment of diseases or disorders characterised by an increased immunological activity.

The number of organ and tissue transplants between different individuals (allotransplants) has increased markedly in recent years. The success of a corresponding transplant is at present determined essentially by the type and extent of the rejection reaction by the recipient's immune system. Transplant rejection has to be inhibited by immunosuppression in every case of allotransplantation.

Immunosuppressive active ingredients, for example corticosteroids, antimetabolites, antilymphocyte serum, anti-IL-2-receptor-antibodies or cyclosporine, are in daily clinical use for the inhibition of transplant rejection as well as for the treatment of a large number of other diseases or disorders. For example, autoimmune diseases and all immune reactions of the acute phase are treated with immunosuppressants (see Pschyrembel, Clinical Dictionary).

A series of peptide-based biotechnologically produced immunosuppressive agents, mostly antibodies, have now been approved for the treatment of various diseases (see Remicade®, Centocor, Inc, Malvern, Pa.; anti-TNF-alpha-antibodies, Essex Pharma GmbH; Orthoclone®, Janssen Pharmaceutica (PTY) Ltd., Aceutica (PTY) Ltd., 15th Road, Halfway House1685; anti-CD3-antibodies, Janssen-Cilag, Saunderton, High Wycombe, Bucks, UK; Keliximab, anti-CD4-antibodies of GalaxoSmithKline; for example).

The available products do, however as a rule, display serious side effects. In particular, the products used for the treatment of transplant rejection lead to a general suppression of all immunological reactions and thus to a weakening of the defence against infectious diseases and an increased danger of the occurrence of malignant diseases. Some of the products also display toxic side effects.

In particular administration of “foreign proteins” to humans, thus of proteins of non-human origin (for example, murine antibodies) leads, in many cases, to undesired side-effects, such as anaphylaxis, sensitisation, increased danger of to thrombosis through the formation of immune complexes and a general cytokine-release syndrome. Furthermore, the immune system of humans, who are subjected to a corresponding treatment, forms antibodies against these foreign proteins, whereby a neutralisation of the same occurs. Foreign proteins can therefore be used only for a very short period of time.

Due to the apoptotic effect on lymphocytes the administration of steroids leads to a dangerous general immunodeficiency in those affected. There is therefore a need for new immunosuppressive active ingredients.

A group of multifunctional proteins have been identified as TIMPs (“tissue inhibitors of metalloproteinases”) due to their ability to inhibit metalloproteinases in the tissue (Corcoran, M. L. and Stedler, W. G., J. Biol. Chem. 270, p. 13453-13459, 1995, hereby incorporated by reference). Zinc-dependent peptidases, including collagenases, gelatinases and stromelysines are called metallo- or matrixmetallo-proteinases (hereinafter “MMP”). These proteinases are able, among other things, to break down the components of the extracellular matrix (hereinafter “ECM”).

Both during tissue development and in the case of pathological disorders building up and breaking down of the ECM structure is of crucial significance. MMPs therefore play an important role in the restructuring of the tissue, for example during morphogensis, angiogensis, repair of tissue and in particular during growth and migration of tumors (Docherty et al., Trends Biotechnol., 10: 200-207, 1992; Matrisian L M, BioEssays, 14: 455-463, 1992; Stetler-Stevenson et al., Annu. Rev. Cell. Biol., 9: 541-573, 1993; Stetler-Stevenson, W. G., J. Clin. Invest., 103: 1237-1241, 1999; DeClerck et al., Adv. Exp. Med. Biol., 425: 89-97, 1997; Jones, J. L. and Walker, R. A., J. Pathol., 183: 377-379, 1997; Westermarck, J. and Kähäri, V. M., FASEB J., 13: 781-792, 1999; Polette, M. and Birembaut, P., Int. J. Biochem. Cell. Biol., 30: 1195-1202, 1998, which are hereby incorporated by reference).

Most MMPs are secreted as a zymogen and activated by further proteinases. The activity of the MMPs is subsequently however regulated primarily by a family of specific inhibitors (the TIMPs). The inhibition takes place through development of irreversible, inactive complexes between TIMPs and MMPs (Cawston et al., Biochem. J., 211: 313-318, 1983, which is hereby incorporated by reference).

To date four TIMP types have been identified and cloned, namely TIMP-1 (Docherty et al., Nature, 318: 66-69, 1985, which is hereby incorporated by reference), TIMP-2 (Boone et al., Proc. Natl. Acad. Sci. USA, 87: 2800-2804, 1990, which is hereby incorporated by reference), TIMP-3 (Apte et al., Genomics, 19: 86-90, 1994; Silbiger et al., Gene, 141: 293-297, 1994; Uria et al., Cancer Res., 54: 2091-2094, 1994; Wilde et al., DNA Cell. Biol., 13: 711-718, 1994, which are hereby incorporated by reference) and TIMP-4 (Greene et al., J. Biol. Chem., 271: 30375-30380, 1996, which is hereby incorporated by reference).

The structural properties of some TIMPs, as well as their mode of operation during MMP inhibition using complex formation have been examined in detail (Tuuttila et al., J. Mol. Biol., 284: 1133-1140, 1998; Bode et al., Cell. Mol. Life Sci., 55: 639-652, 1999; Gomis-Rüth et al., Nature 389: 77-81, 1997, which are hereby incorporated by reference). An equilibrated balance between MMPs and TIMPs is of great importance physiologically. For this, the quantities of TIMPs are regulated by steroids, growth factors and cytokines, such as for example IL-1, IL-6, IL-10, leukemia-inhibitory factor, neurotrophic factor, oncostatin M, TNF-alpha and epidermal growth factor (Fabunmi et al., Biochem. J., 315: 335-342, 1996; Roeb et al., FEBS Letters, 349: 45-49, 1994; Nemoto et al., Arthritis Rheumatism, 39: 560-566, 1996; Lotz, M. and Guerne, P. A., J. Biol. Chem., 266: 2017-2020, 1991; Hosono et al., FEBS Letters, 381: 115-118, 1996; Lacraz et al., J. Clin. Invest., 96: 2304-2310, 1995; Shingu et al., Clin. Exp. Immunol., 94: 145-149, 1993, which are hereby incorporated by reference).

As well as having in common the activity of proteinase-inhibition, every TIMP also has, however, additional properties that differ from TIMP to TIMP. TIMP-1 is primarily active in B cells and B cell lymphomas, while the expression of TIMP-2 is limited to T cells. TIMP-1 and -2 have a proteinase-inhibitor domain at the NH₂-end and a growth factor domain at the COOH end. The proteinase inhibitors act however on different proteinases. TIMP-2 inhibits MMP2, a proteinase that specifically digests basal membrane collagen IV (the collagen of the basal membrane of vessels). The MMP2 function is essential for lymphocytes as it makes it possible for these to emerge from the vessel wall.

TIMP-1 inhibits MMP-1, -3 and -9, proteinases that primarily digest collagen III, but have no influence on vessel walls.

TIMP-1 and -2 have an overall homology of roughly 40%, the greatest homology being in the area of the domains responsible for the proteinase inhibitor activity (Fernandez-Catalan et al., EMBO J., 17, 5238-48, 1998; Greene et al., J. Biol. Chem., 271, 30375-80, 1996; Hayakawa et al., J. Cell. Sci., 107, 2373-9, 1994, which are hereby incorporated by reference).

Both the over-expression of TIMP-1 in non-Hodgkin's lymphomas (hereinafter “NHLs”) and the correlation with the clinical aggressiveness of the disease have been described in the state of the art (Kossakowska et al., Blood, 77: 2475-2481, 1991, which is hereby incorporated by reference). It was furthermore known that the movement of the lymphocytes is determined by the equilibrium between the MMPs and TIMPs produced by these cells (Johnatty et al., J. Immunol., 158: 2327-2333, 1997; and Borland et al., J. Biol. Chem., 274: 2810-1815, 1999, which are hereby incorporated by reference). Finally, it was known that TIMP-1 induces the differentiation of the B cells (Guedez et al., J. Clin. Invest., 102: 2002-2010, 1998; Guedez et al., Blood, 92: 1342-1349, 1998, which are hereby incorporated by reference).

The state of the art contains experiments on animals that show that TIMP-2 can be used for the treatment of allergic inflammations, in particular skin inflammations or atopic dermatitis (JP 2000086533, which is hereby incorporated by reference). Recently it was also reported that TIMP-2 has the ability to induce apoptosis in activated peripheral T cells. No apoptosis was induced in non-stimulated T cells. In this connection it was ascertained furthermore that a TIMP-2 specific effect was involved. In these studies, TIMP-1 had no apoptotic effect on activated T cells (Lim et al., PNAS, Vol. 878 (1999), p. 522-523, which is hereby incorporated by reference).

### BRIEF SUMMARY OF THE INVENTION

The present invention provides a use of TIMP-1, a TIMP-1 analogue, a fragment of TIMP-1 or a TIMP-1 analogue with immunosuppressive activity, or a nucleic acid which encodes TIMP-1, a TIMP-1 analogue, a fragment of TIMP-1 or a TIMP-1 analogue with immunosuppressive activity for the preparation of a pharmaceutical composition for the treatment of diseases or disorders, which are characterised by an increased immunological activity. The present invention also provides a TIMP-1 analogue, which is a natural or recombinant allelic variant of TIMP-1 and displays a homology of at least 50%, preferably at least 70% with the TIMP-1 amino acid sequence. The present invention also provides a TIMP-1 analogue, which is a natural or recombinant allelic variant of TIMP-1 and displays a homology of, at least 80%, preferably at least 95% with the TIMP-1 amino acid sequence.

The present invention also provides a use according to the present invention, in which the fragment has a length of at least 3, preferably at least 5 or 10 amino acids.

The present invention also provides a use according to the present invention in which the nucleic acid coding for TIMP-1, for the TIMP-1 analogue or for one of the fragments with immunosuppressive activity is operatively linked to a sequence which can effect an expression of the sequence. The present invention also provides for a use in which the nucleic acid is part of an expression construct which is suitable for the transformation of target cells in the patient.

The present invention provides a use according to the present invention, in which the TIMP-1, the analogue, their fragments or a corresponding nucleic acid is used for the treatment of immune diseases, which are mediated by Th1 cells, abnormally activated Th2 cells, activated CD8 or CD4 cells, activated eosinophilic granulocytes, mast cells and/or abnormally secreting cells (such as e.g. epithelial cells of the nose and of the bronchial system).

The present invention provides a use according to the present invention, in which the TIMP-1, the analogue, the fragment or the nucleic acid is used for the treatment of multiple sclerosis, Crohn's disease, acute and chronic graft-versus-host diseases, acute transplant rejection, type 1 diabetes mellitus, rheumatoid arthritis, Lyme arthritis, reactive Yersinia-induced arthritis, post-streptoccocus cardiac valve and myocardial diseases, hepatitis C-induced chronic hepatitis, Hashimoto's thyroiditis, Grave's disease, primary sclerosing cholangitis, helicobacter pylori-induced gastrititis, cerebral malaria, contact dermatitis, aplastic anaemia, immunologically provoked abortions, bronchial asthma, sunburn, hay fever and allergic diseases.

The present invention also provides a use according to the present invention, in which TIMP-1, the analogue, their fragments or a corresponding nucleic acid is present as an injection solution, infusion solution, nose drops or nose sprays, drops, mouth wash, inhalants, tablets, plaster or cream.

The present invention also provides a use according to the present invention, in which the TIMP-1, the analogue, their fragments or a corresponding nucleic acid is used for the in-vitro treatment of tissue before transplantation.

The present invention also provides a method for production of a medicament for the treatment of diseases or disorders which are characterized by an increased immunological activity, in which TIMP-1, a TIMP-1 analogue, their fragments or a nucleic acid coding for the same, is mixed or coupled with a pharmaceutically compatible carrier.

The present invention also provides a rinsing solution for transplants, comprising TIMP-1, a TIMP-1 analogue, a fragment thereof or a nucleic acid encoding the same and a pharmaceutically compatible carrier.

### BRIEF DESCRIPTION OF THE DRAWINGS

FIG. 1 shows results of the allogeneically activated, mixed lymphocyte culture, the inhibition of the lysis by TIMP is represented as a percentage amount of specific chromium release.

FIG. 2 shows results of the FACS analysis of the influence of rhTIMP-1 on the apoptosis of activated lymphocytes.

FIG. 3 shows the DNA synthesis rate of mixed allogeneically stimulated lymphocyte cultures in the presence and absence of rhTIMP-1.

FIG. 4 shows the DNA synthesis rate of mixed allogeneically stimulated lymphocyte cultures in the presence and absence of rhTIMP-1.

FIG. 5 shows the DNA synthesis rate of mixed allogeneically stimulated lymphocyte cultures in the presence and absence of rhTIMP-1 after division of the lymphocytes into subpopulations.

FIG. 6 shows induction of RNA expression of the Th2 cell-specific transcription factor Gata-3 and reducing the transcription factor TZFP specific for T-cell activation by rhTIMP-1 in allogeneic lymphocyte cultures.

FIG. 7 shows the inhibition of the cytotoxic effect of perforin on a human T-cell line (Jurkat cells) using rhTIMP-1.

FIG. 8 shows the inhibition of the intracellular Ca²⁺ influx induced by perforin by rhTIMP-1.

FIG. 9 shows the inhibition of the intracellular Ca²⁺ influx mediated by eotaxin in eosinophilic granulocytes with subsequent inhibition of the secretion of the toxic molecule EDN (“eosinophile derived neurotoxin”).

### DESCRIPTION OF THE NUCLEIC ACID SEQUENCES

SEQ ID NO: 1 sets forth a amino acid sequence of a melanoma-associated nonapeptide.

SEQ ID NO: 2 sets forth a nucleic acid sequence of a sense Gata-3 primer.

SEQ ID NO: 3 sets forth a nucleic acid sequence of a antisense Gata-3 primer.

SEQ ID NO: 4 sets forth a nucleic acid sequence of a Gata-3 probe.

SEQ ID NO: 5 sets forth a nucleic acid sequence of a sense TZFP primer.

SEQ ID NO: 6 sets forth a nucleic acid sequence of a antisense TZFP primer.

SEQ ID NO: 7 sets forth a nucleic acid sequence of a TZFP probe.

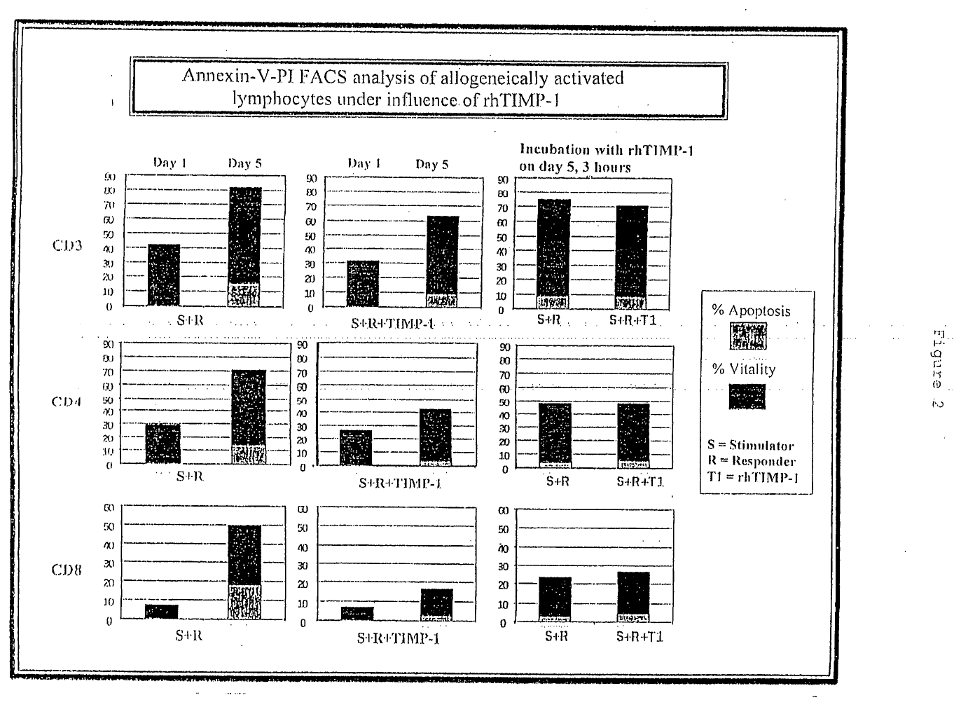

SEQ ID NO: 8 sets forth a nucleic acid sequence of a TIMP-1 open reading frame from FIG. 2 of Docherty et al., Nature, 318:66-69, 1985.

SEQ ID NO: 9 sets forth an amino acid sequence of a recombinant human (hereinafter “rh”) TIMP-1.

SEQ ID NO: 10 sets forth an amino acid sequence of a rhTIMP-2;

### DETAILED DESCRIPTION OF THE INVENTION

The present invention surprisingly discloses that TIMP-1 also has an immunosuppressive activity. The present invention thus relates to the use of

(a) TIMP-1;

(b) a TIMP-1 analogue;

(c) a fragment of (a) or (b) with the same immunosuppressive activity as TIMP-1; or

(d) a nucleic acid which encodes one of the peptides named in (a) to (c); for the preparation of a pharmaceutical composition for the treatment of diseases or disorders characterised by an increased immunological activity.

Within the framework of the present invention a protein with the amino acid sequence disclosed in FIG. 2 of the publication of Docherty et al. (Nature, 318: 66-69, 1985, which is hereby incorporated by reference) is called TIMP-1. On the other hand, the TIMP-1 analogues are variants of TIMP-1 which occur naturally or which are made using chemical or recombinant processes, which display differences in the amino acid sequence but essentially the same immunosuppressive activity. Corresponding analogues have, compared with the TIMP-1 amino acid sequence, a degree of homology of at least 50%, preferably at least 70%. According to a particularly preferred version the TIMP-1 analogues have a degree of homology of at least 80%, in particular at least 95% with the TIMP-1 amino acid sequence. The degree of homology is determined by writing the two sequences one above the other, four gaps being possible over a length of 100 amino acids, in order to achieve the greatest possible similarity of the sequences to be compared (see also Dayhoff, Atlas of Protein Sequence and Structure, 5, 124, 1972, which is hereby incorporated by reference). The percentage of the amino acid residues of the shorter of the two amino acid chains, which lies opposite identical amino acid residues on the other chain, is then established.

A large number of processes are known in the state of the art by means of which proteins and peptides are modified, for example by derivatization of individual groups of the amino acids or by binding to macromolecules (e.g. PEG and PEG derivatives or other proteins for the production of fusion proteins). Through the derivatization the peptides are said to acquire advantageous properties (improved stability, etc). Corresponding derivatives of the above-named peptides are covered by the present invention and are likewise called TIMP-1 analogues.

A TIMP-1 analogue has, for the purposes of the present invention, the same immunosuppressive activity as TIMP-1 if it inhibits the T-cell-mediated cytotoxicity in a mixed lymphocyte culture measured using the chromium-release detection procedure at the rate of at least 65%, preferably at least 75%, in particular at least 85%, (inhibition by TIMP-1 being taken as 100%). The mixed lymphocytes culture can be carried out as in the following examples (see Example 1) and according to processes known in the state of the art (see Kägi et al., Science, 265, 528-530, 1994; and Lowin et al., Nature, 370, 650-652, 1994, which is hereby incorporated by reference).

Within the framework of the present invention fragments of TIMP-1 or of the analogues can naturally also be used. The fragment can have any size as long as it has the same immunosuppressive activity as TIMP-1. The fragment has a length of at least 3 amino acids, preferably a length of at least 5 amino acids, a length of 10 to 20 amino acids being particularly preferred.

The TIMP-1 analogues and fragments discussed here can be created by a person skilled in the art, for example, by recombinant production after introducing substitutions or deletions in the known TIMP-1 nucleic acid sequence. Alternatively, the analogues can be produced by chemical synthesis. In each case, it is a simple matter to determine the immunosuppressive activity of the analogues. Corresponding TIMP-1 analogues and fragments are thus directly available to a person skilled in the art.

According to another embodiment, the present invention relates to the use of a nucleic acid which codes for TIMP-1, a TIMP-1 analogue or a fragment thereof for the production of a pharmaceutical composition for the treatment of diseases or disorders which are characterised by an increased immunological activity. In the state of the art various processes are known by means of which nucleic acids are used directly or in combination with a carrier for the in-vitro and in-vivo transformation of cells and thus for the treatment of diseases.

For the targeted gene transfer into eukaryotic cells of the hematopoietic system, vectors have been used for example which are based on retroviruses (see Dao et al., Int. J. Mol. Med., 1, 257-264, 1998; and Pollok et al., Curr. Op. Mol. Ther., 1, 595-604, 1999, which are hereby incorporated by reference). Vectors based on SV40 and HSV have also already been used in the state of the art in vitro and in vivo for gene transfer into specific eukaryotic cells (Strayer, D. S., J. Cell. Physiol., 181, 375-384, 1999; and Stevenson et al., Semin. Hemol., 36, 38-42, 1999, which are hereby incorporated by reference).

The present invention comprises the use of corresponding processes based on nucleic acids during the medical application of TIMP-1, its analogues and fragments. The nucleic acids can for example be RNA or DNA, a DNA being preferably used.

According to this aspect the nucleic acid encoding TIMP-1, the TIMP-1 analogue or one of the fragments with immunosuppressive activity is preferably operatively linked with a regulatory sequence which can effect the expression of the coding sequence. According to a particularly preferred embodiment, a regulatory sequence is used that allows expression of the coding sequences exclusively in selected target cells.

Conditions or disorders that are characterised by an increased immunological activity are immediately identifiable by a medical doctor. A feature of a corresponding disease or disorder is for example the lysis of organs or cells occurring naturally in the body by lymphocytes. Diseases characterised by an increased immunological activity may for example be autoimmune diseases. Alternatively or additionally conditions or disorders characterised by an increased immunological activity can be identified by excessive release of mediator substances. Examples of corresponding conditions are allergic diseases in which inter alia mediators, such as cytokines, etc., are released by lymphocytes, thereby causing the disease (hay fever, asthma, as non-limiting examples).

The present analysis of the immunosuppressive effect of TIMP-1 indicates that, unlike TIMP-2, TIMP-1 does not induce T cell apoptosis but could have an activating effect on inhibitory T cells (e.g., CD4, Th2 cells, which express the transcription factor Gata-3) and/or inhibiting effect on activating T cells (for example CD4, Th1 cells, and activated, TZFP-positive lymphocytes).

Further, TIMP-1 appears to reduce or block the degranulation of activated cells and thus the spillage of toxic substances. For example, secretion of eosinophile derived neurotoxin (hereinafter “EDN”) from activated, eosinophilic granulocytes (from patients with allergic rhinitis) is shown in the examples. Unlike apoptosis induction by TIMP-2 the results of the present application show a protective effect on the examined cell populations. With the inhibition of activated cells (effectors), TIMP can have a protective effect on the target cell population (inhibition of apoptosis) by inhibiting toxic substances, such as e.g. perforin.

Active ingredients which have an immunosuppressive effect without inducing apoptosis in T cells are pharmacologically of particular interest as they offer the possibility of achieving immunosuppression without inducing a general and lasting immune deficiency.

Use of the TIMP-1, the TIMP-1 analogue, their fragments or the corresponding nucleic acid according to the invention thus includes inter alia the use for the treatment of immune diseases which are mediated by TH1 cells, abnormally activated Th2 cells, activated CD8 or CD4 cells, activated eosinophilic granulocytes, mast cells and/or abnormally secreting cells (such as, for example, epithelial cells of the nose and of the bronchial system).

The TIMP-1, the TIMP-1 analogue, their fragments, or a corresponding nucleic acid can be used in particular for the production of a pharmaceutical composition for the treatment of multiple sclerosis, Crohn's disease, acute and chronic graft-versus-host diseases, acute transplant rejection, type I diabetes mellitus, rheumatoid arthritis, Lyme arthritis, reactive Yersinia-induced arthritis, post-streptococcus cardiac valve and myocardial diseases, Hepatitis C-induced chronic hepatitis, Hashimoto's thyroiditis, Grave's disease, primary sclerosing cholangitis, helicobacter pylori-induced gastrititis, cerebral malaria, contact dermatitis, aplastic anaemia, immunologically provoked abortions, bronchial asthma, allergic skin conditions, sunburn or hay fever.

It was established that TIMP-1 is over-expressed in lymph nodes of patients with Hodgkin's disease. The protein according to the invention should therefore preferably not be administered to patients with B cell lymphomas.

According to the invention TIMP-1, the TIMP-1 analogue, their fragments or a corresponding nucleic acid can be applied in any of the known dosage forms. Application in the form of an injection solution, infusion solution, nose drops, nose spray, drops, mouthwash, inhalants, tablets, plaster or cream is preferred. Accordingly the present invention also relates to methods for the production of a medicament for the treatment of diseases or disorders which are characterised by an increased immunological activity. These can be in particular an infusion solution, injection solution, a tablet, a plaster or a cream. For preparing the same, TIMP-1, a TIMP-1 analogue, their fragments or a nucleic acid coded thereof are mixed with a pharmaceutically compatible carrier.

According to an alternative aspect TIMP-1, the TIMP-1 analogue, their fragment or a corresponding nucleic acid can be used for the in-vitro treatment of transplant tissue or organs before transplantation. For this, a rinsing solution for transplants is made which includes the named active ingredient. This procedure achieves a so-called T-cell purging.

When evaluating the statistical analyses of the following examples, either the Mann-Whitney test (Examples 1 to 3), in which a P value of less than 0.05 was rated as a significant difference, or a simple establishment of the standard deviations (Examples 4 to 9) were used.

### EXAMPLE 1

### Influence of TIMP-1 on Mixed and Autologous Lymphocyte Cultures

The mixed lymphocyte cultures were carried out according to methods known in the state of the art (Kägi et al., Science, 265: 528-530, 1994; and Lowin et al., Nature, 370: 650-652, 1994, which are hereby incorporated by reference).

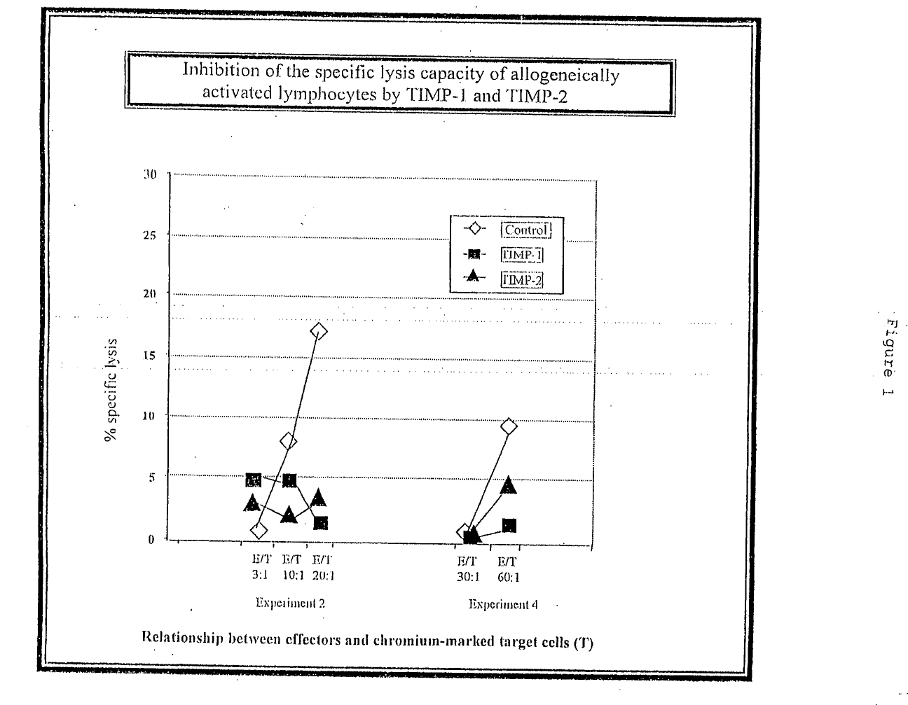

In brief, blood from various non-histocompatible, healthy donors (stimulators and responders) was obtained and treated with Ficoll® (Pfizer, New York, N.Y.). The cells of the interphase were isolated and washed twice. Mononuclear cells acting as stimulators were irradiated with 30 Gy. The stimulation was carried out in a test with CD14-enriched (MACS, CD14 Microbeads, Miltenyi Biotech, Bergisch-Gladbach, Germany) and irradiated cells. For this, 8×10⁶ irradiated stimulators and 2×10⁷ living responder cells were incubated for 5 days in 15 ml RPMI 1640 medium with 2 mM glutamine and 10% FCS in 50 ml Falcon test tubes accompanied by regular agitation (once daily) (pH 7.2, 37° C., 5% CO₂, high humidity). On the 5^{th} day fresh, non-irradiated stimulator cells were incubated in a concentration of 2×10⁶ cells per 100 μl for 90-120 minutes with 100 μCi on sodium [⁵¹Cr]-chromate (100 μl volume, specific activity 472.220241 mCi/mg, NEN; pH 7.2, 37° C., 5% CO₂, and high humidity). The labelled cells were washed three times with 10% FCS in 1640 RPMI medium. Microtitre plates were filled with 150 μl marked stimulator/target (E) cells and responder/effector (E) cells in the stated E:T quantity in the presence of rhTIMP-1 or rhTIMP-2 or a vehicle as a control. The microtitre plates were incubated for 4 hours (pH 7.2, 37° C., 5% CO₂ high humidity) and centrifuged for 5 minutes at 200×g. Aliquot parts of the supernatants were then examined for radioactivity. Maximum chromium release was measured after lysis of the marked stimulator cells (corresponds to the “target cells” or T) using Triton X 100 treatment of the wells. The spontaneous chromium release was measured on target cells that had only been maintained in medium. The results (see FIG. 1) show the percentage amount of specific chromium release as an average of triple measurements (experimental chromium release (cpm) minus spontaneous chromium release (cpm)×100 divided by the maximum chromium release (cpm) minus spontaneous release).

Alternatively, a test was carried out, wherein autologous (HLA-A2-positive) PHA-stimulated lymphocytes were incubated with a melanoma-associated nonapeptide (IMDQVPFSV, a gp100 peptide which has been altered in position 2, in order to obtain improved affinity compared with the native peptide for HLA-A*0201-binding sites; see Parkhurst, M. R., J. Immunol., 157: 2539-2548, 1996, which is hereby incorporated by reference) as stimulator/target cell. After triple stimulation by nonapeptide-presenting cells (autologous to the effector-lymphocytes) incubation was carried out with lymphocytes as responder/effector cells, otherwise using the parameters given for the mixed lymphocyte culture.

FIG. 1 shows as an example results that were obtained carrying out the chromium release detection after mixed lymphocyte culture. As this figure clearly shows, both rhTIMP-1 and rhTIMP-2 inhibit the T-mediated cytotoxicity to various target cells after only 3 hours in the culture and 4 hours assay duration. This effect was most strongly pronounced at higher E:T-ratios and reached values of between 84% and 89% inhibition of the controls (without cytokine).

### EXAMPLE 2

### Influence of rhTIMP-1 on the Apoptosis of Activated Lymphocytes

In order to examine possible causes for the reduced lysis capacity of the allogeneically stimulated lymphocytes, the vitality of these cells was checked by means of their capacity to proliferate, and their apoptosis behaviour.

For this, mixed mononuclear cells were isolated and stimulated analogously to the protocol for mixed lymphocyte cultures. 5 days after the start of the trial 1×10⁵ cells per trial condition were dyed (dyeing with lymphocyte-subpopulation-specific antibodies against CD3, CD4 or CD8) with Annexin V and propium iodide using standard methods (BD FACSCalibur™ System, Becton Dickinson), in order to be able to distinguish between apoptotic cells and dead cells. The cell suspension was left at 4° C. for one hour in the dark and analysed using a FACS analyser (FACS Calibur™, BD Biosciences, San Jose Calif., USA). Further evaluation was carried out by means of CellQuest™ and Paint-A-Gate 3.0 Software (BD Biosciences, San Jose Calif., USA) on a Macintosh PC.

The results are summarised in FIG. 2 and show that rhTIMP-1 has no apoptosis-promoting effect on activated lymphocytes, not even on a subpopulation of the lymphocytes.

### EXAMPLE 3

### Influence of TIMP-1 on the DNA-Synthesis Rate in Mixed Lymphocyte Cultures

In 8 trials the DNA synthesis rate of the mixed lymphocyte cultures was measured by ³H-thymidine absorption of the cells.

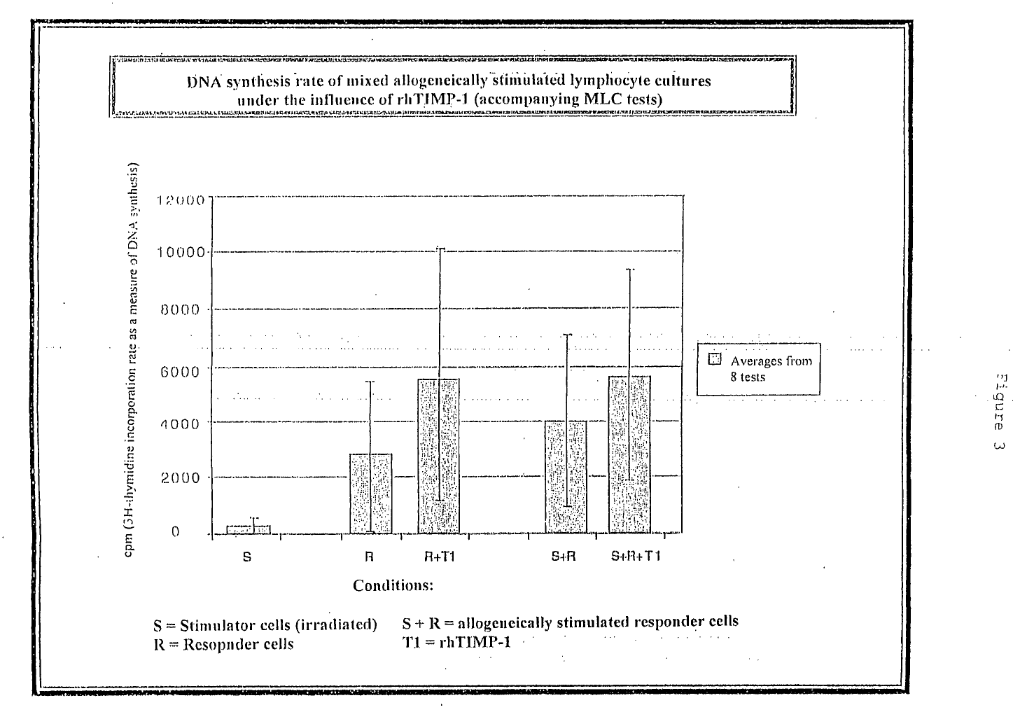

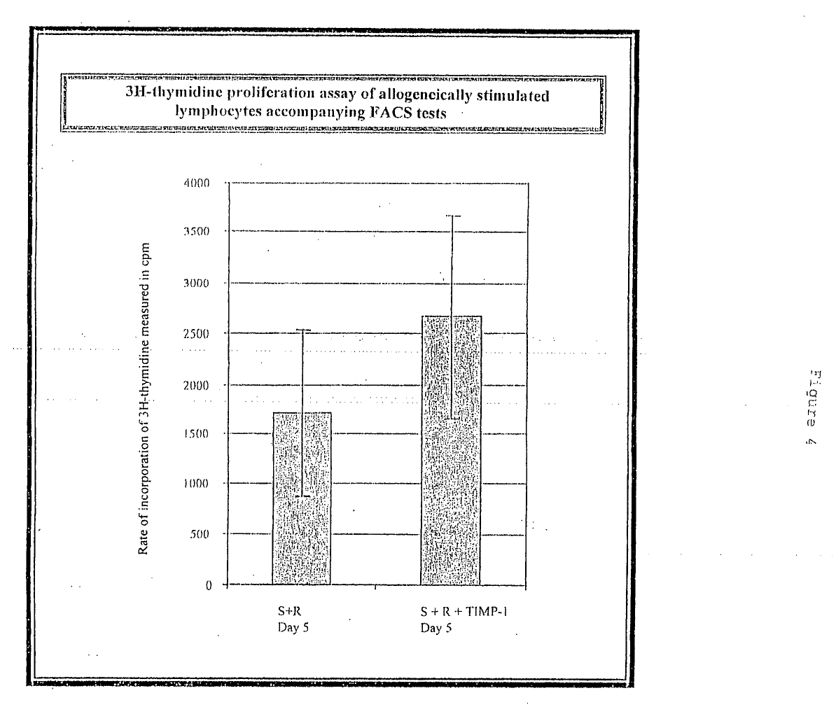

The presence of TIMP-1 did not lead to a significant drop in the thymidine absorption. The trend was more towards an increase in the proliferation, which was however not statistically significant. These results do show however that no induced cell death could be observed in the decisive cell population (see FIGS. 3 and 4).

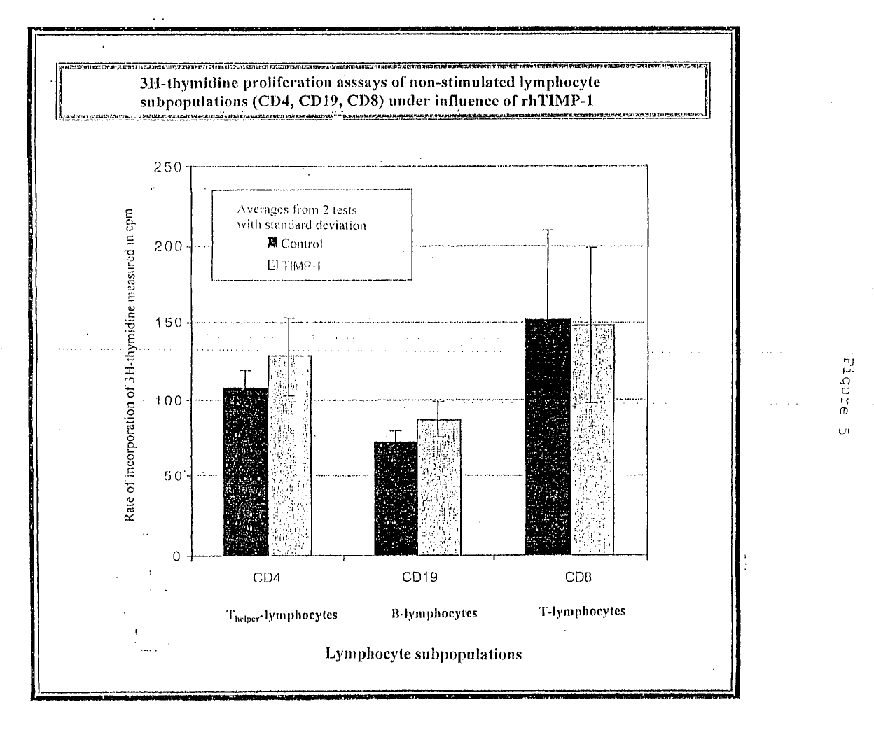

Finally, the ³H-thymidine absorption of lymphocytes from mixed lymphocyte culture was determined, the lymphocytes were divided beforehand into subpopulations by FACS separation. This evaluation (FIG. 5) also clearly shows that TIMP-1 does not have an apoptotic effect on lymphocytes.

### EXAMPLE 4

### Influence of TIMP-1 on the RNA Synthesis of the Transcription Factors Gata-3 and TZFP

This example describes the analysis of the influence of TIMP-1 on the quantitative RNA synthesis rate of the transcription factors Gata-3 and TZFP (“testis zinc finger protein” or “repressor of Gata-3”) in mixed allogeneic lymphocyte cultures.

In allogeneically stimulated lymphocyte culture, a co-ordinated interplay between CD4-Th1, as well as CD8 cells is essential. CD4-Th2 cells play either a subordinate—or even an inhibiting role in this process. A feature by which thoroughly activated T-lymphocytes can be recognised is the transcription factor TZFP, which as a repressor protein binds and inactivates Gata-3. Gata-3 by contrast is found in differentiated cells almost exclusively in CD4-Th2 cells and thus should rather decrease during an allogeneically stimulated condition.

The lymphocytes which are to be analysed were, as described in example 1, obtained from the blood of healthy normal people and cultured over 5 days. At the start of the trial, hour 0, as well as on day 5 the cells were exposed to various conditions (E=effector alone; E+T1 6 h=effector incubated on day 5 for 6 hours with rhTIMP-1 [500 ng/ml]; E+T1 5T=effector with rhTIMP-1 [500 ng/ml]; from hour 0 until day 5). At the given times the cells were spun down, and RNA was isolated according to standard protocols (RNAzol). 1 μg of RNA per mixture was transcribed to cDNA by means of primers (random hexamers) and using Superscript™ II reverse transcriptase (Invitrogen Corp., Carlsbad, Calif., USA). The cDNA was diluted 1:200 μl with ddH₂O.

Of this cDNA 5 μl was used in a PCR. The quantification of the mRNA was carried out by the real-time fluorescence detection method. The PCR took place in the ABI prism 7700 Sequence Detector (PE Biosystems, Foster City, Calif.). Primers and probes were used, specific to GAPDH or 18S, as control, as well as Gata-3 and TZFP, which were labelled at the 5′ end with VIC (GAPDH, 18S), or FAM (all other specimens) and at the 3′ end with TAMRA which serves as quencher.

The 5′-3′ nuclease activity of the Taq-polymerase cuts off the sample and thus leads to the release of the fluorescent dyes (FAM, VIC), which can be measured by the laser detector of the PCR cycler. After a threshold value has been exceeded, the fluorescence obtained is proportional to the quantity of the generated PCR product. Every studied microtitre plate with 96-wells contained 12 standard samples (dilution series of resting lymphocytes).

The relative gene expression of every sample was calculated using the standard curve for every condition. The constantly expressed genes such as GAPDH and 18S-RNA served as additional controls for the calculation, as well as for the comparison of the quality of the cDNA.

The following nucleotides were used as PCR primers and as probe for the transcripts to be studied (corresponding primers and probes for GAPDH and 18S-RNA are commercially available):

| | |
|---|---|
| Gata-3: | |
| Primer | 5′-3′ direction: |
| | 5′gga-cga-gaa-aga-gtg-cct-caa-3′ |
| Primer | 3′-5′ direction: |
| | 5′tgg-gac-gac-tcc-agc-ttc-a-3′ |
| Probe: | 5′agg-tgc-ccc-tgc-ccg-aca-gc-3′ |
| TZFP: | |
| Primer | 5′-3′ direction: |
| | 5′ata-gca-ccc-cca-cca-ctg-g-3′ |
| Primer | 3′-5′ direction: |
| | 5′ggc-att-tag-gga-cag-tgg-ga-3′ |
| Probe: | 5′cag-gag-gtc-tgg-cgg-gaa-cag-agg-3′ |

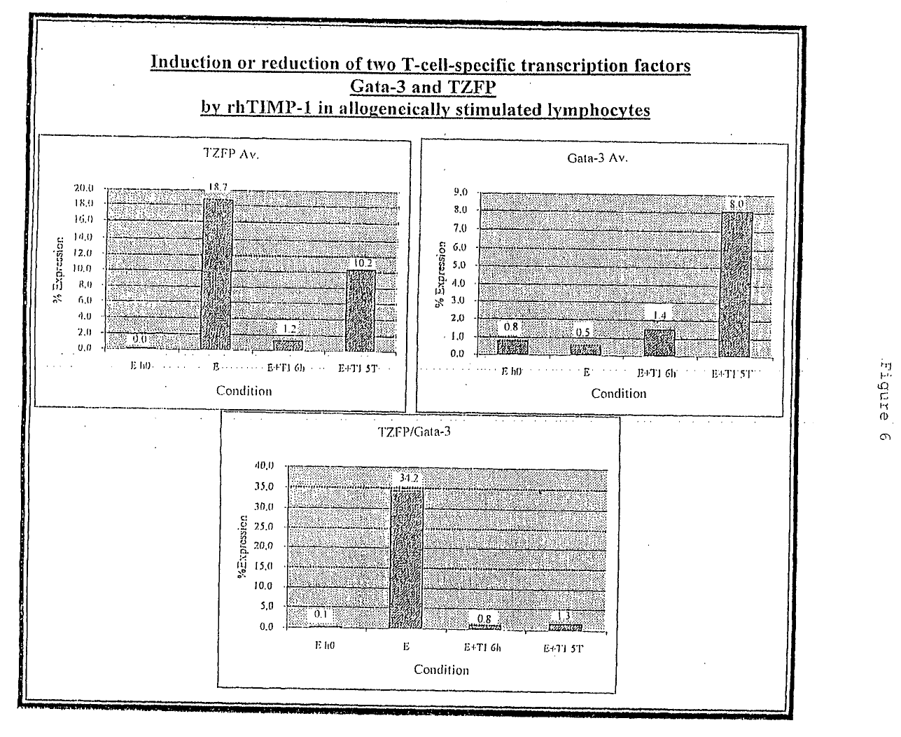

The results are presented in FIG. 6 and show that the incubation of allogeneically stimulated lymphocytes with rhTIMP-1 leads to a clear reduction of the expression of the transcription factor TZFP. Under normal stimulation conditions, this transcription factor clearly increases.

The gene expression of the Th2-specific transcription factor Gata-3 in contrast is clearly increased by rhTIMP-1, which suggests an increased presence of Th2 cells which are not detectable under normal stimulation conditions.

These studies thus suggest that the influence of rhTIMP-1 on these two transcription factors comprises effects on various lymphocyte subpopulations which are brought about by the immunosuppressive effect of TIMP-1.

### EXAMPLE 5

### Influence of TIMP-1 on the Perforin-Induced Apoptosis/Necrosis of Human T-Cells

In this example the influence of TIMP-1 on the perforin-induced apoptosis/necrosis of the human Jurkat T-cell line was studied.

Perforin is a glycoprotein which is secreted from activated cytotoxic cells (CTLs, NK cells) and which in target cells through the formation of pores into the membrane leads to cell death (necrosis) of same. A first consequence of this pore formation is the influx of ions, e.g. calcium, into the target cells from outside.

FACS analysis: The propidium iodide (PI) measurement of dyed cells and their analysis by means of FACS is based on this principle, as PI can be adsorbed solely into dead cells or cells with a membrane which is no longer intact.

To this end the Jurkat T-cell line was incubated in a concentration of 1×10⁶ cells/ml with 20 ng/ml perforin for 4 hours at 37° C. and then dyed with PI using standard methods (FACSCalibur™, Becton Dickinson). Instead of perforin, the same volume of 1×PBS was used as a control.

The mixtures called “TIMP-1-condition” were pre-incubated with 500 ng/ml rhTIMP-1 for 1 hour at 37° C. and then pipetted to produce the perforin or perforin and rhTIMP-1 in the stated concentrations and were pipetted together to the cells at the start of the 4-hour incubation.

Appropriate tests were carried out by adding Granzyme B 100 ng/ml to the respective conditions for the targeted induction of apoptosis and analysed by standard dyeing with Annexin-V, or with Yopro-I together with propidium iodide. In both cases further evaluation was by means of CellQuest™ and Paint-A-Gate 3.0 Software (BD Biosciences, San Jose Calif., USA) on a Macintosh PC.

Trypan Blue Dye: Trypan Blue is a dye which cannot penetrate intact cell membranes and as such only dyes blue cells which are either dead or which have holes in the membrane. The cells were incubated according to the aforementioned conditions (control, perforin, TIMP-1, perforin+TIMP-1) for 30 minutes at 37° C. and then stained with this dye. For this, 50 μl cell suspension (1×10⁶ cells/ml) were mixed with 450 μl Trypan Blue and evaluated under the microscope.

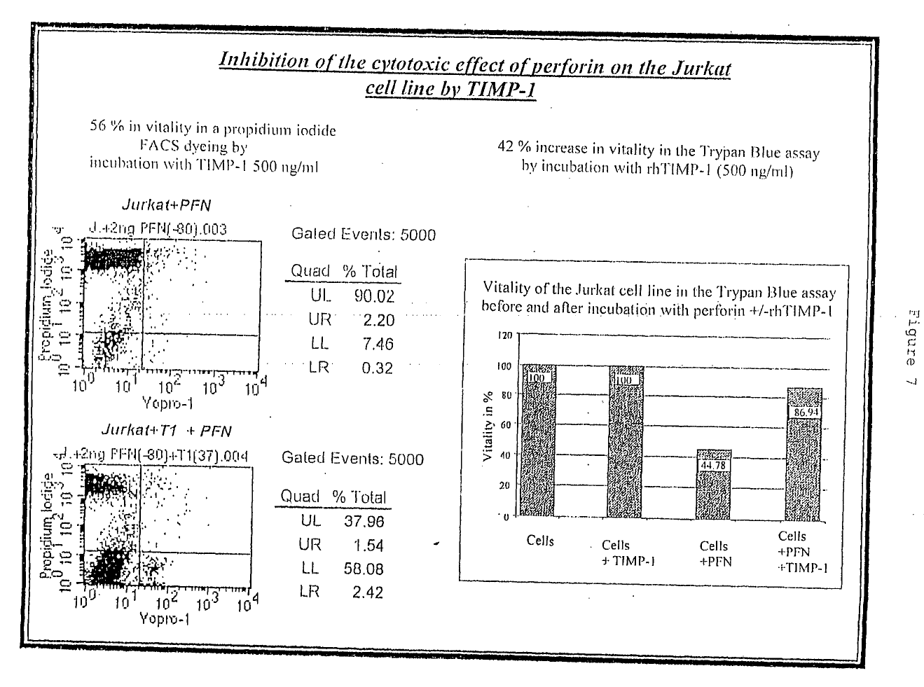

The results are presented in FIG. 7 and show that rhTIMP-1 is able to inhibit the pore formation induced by perforin in the membrane of the Jurkat cell line and the subsequent necrosis induction. In a total of 20 analyses rhTIMP-1 was able in all mixtures, to inhibit 10 to 56% of the necrosis induced by perforin as well as the apoptosis induced by perforin+Granzyme B. These results were also confirmed by the Trypan Blue assay.

### EXAMPLE 6

### Influence of TIMP-1 on the Calcium Influx into a Human T-Cell Line (Jurkat Cells)

In this example the influence of TIMP-1 on the calcium influx induced by the cytotoxic-lytic effect of human perforin in human T-cells (Jurkat) was studied.

As described in Example 5, the glycoprotein perforin induces the formation of holes in the membranes of human cells, which leads to the influx of calcium into the cells from outside (from the buffer).

This calcium influx can be represented by staining of the cells with the dye Fura-2, which is accumulated within cells and which upon influx of Ca²⁺ into the cells binds the Ca²⁺ and at that moment increases its fluorescence properties. The difference in fluorescence between bound and unbound calcium is measured in a fluorescence spectrometer.

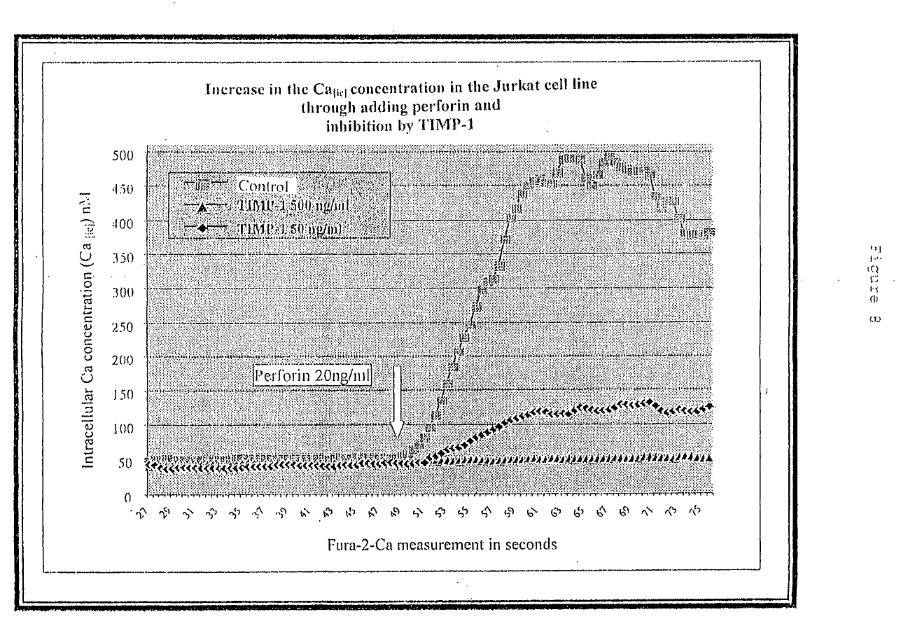

The results are presented in FIG. 8 and show that rhTIMP-1 was able to inhibit the perforin-triggered Ca influx into the cells.

### EXAMPLE 7

### Influence of TIMP-1 on the Eotaxin-Induced Calcium Influx into Eosinophilic Granulocytes

In this example the influence of TIMP-1 on the eotaxin-induced calcium influx into eosinophilic granulocytes was studied. The subsequent changes in the secretion of the toxic protein of the eosinophilic granulocytes, EDN, were also studied.

Eosinophilic granulocytes of allergic patients are also an example of an activated cell of the immune system with corresponding hyperfunction. The induction of this secretion by eotaxin and IL-5 is described precisely in the literature (Fusjisawa, T. et al., J. Allergy Clin Immunol, 2000, which is hereby incorporated by reference).

To check the function of rhTIMP-1 on these cells, we firstly isolated granulocytes from heparinized blood of patients with allergic rhinitis following standard Milteniy protocols, i.e. by means of Ficoll® (Phizer Inc., New York, N.Y.), purification and further enriching the eosinophilic granulocytes from this population by means of CD16 depletion.

A part of the 90% pure population of eosinophilic granulocytes was then either dyed with Fura-2 and subjected to calcium-fluorescence measurement under the action of eotaxin or incubated in a microtitre plate with 96 wells with the cytokine/chemokine given below (eotaxin 1×10⁷ mol/1; IL-5 2,5 ng/ml; eotaxin and IL-5 ±rhTIMP-1). After various periods the supernatants of these cultures were frozen and then examined for the presence of the EDN protein, using an ELISA.

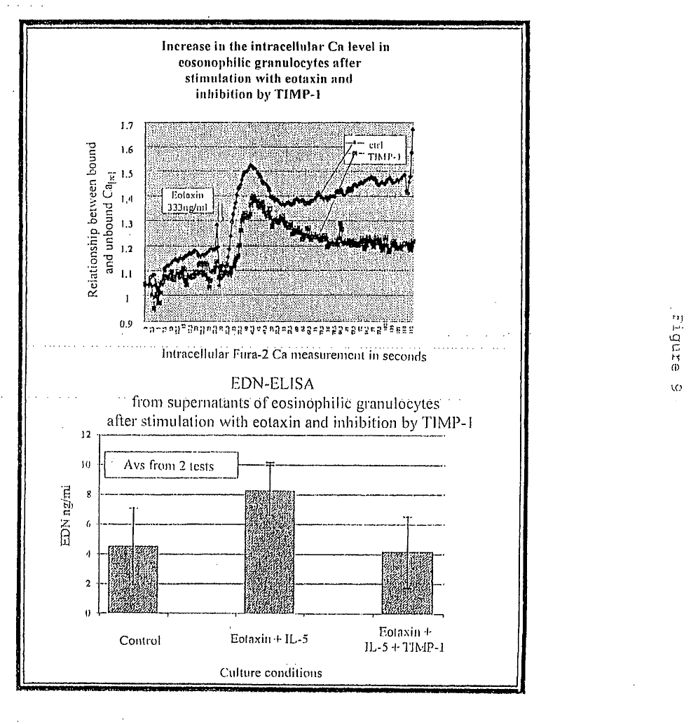

The results are presented in FIG. 9, averages from three different times being shown. The pre-incubation of the cells with rhTIMP-1 for 1 hour at 37° C. led to a partial inhibition of the Ca²⁺ influx into the cells triggered by eotaxin. rhTIMP-1 inhibited the secretion of EDN in the same cell population down to the control value.

What is claimed:

1. A method of treating a patient having a disease, wherein said disease is characterised by increased immunological activity, comprising:
(a) providing an amino acid molecule having an amino acid sequence selected from the group consisting of a TIMP-1, a TIMP-1 analogue, and fragments of either that are capable of immunosuppressive activity; and
(b) treating said patient having said disease with said amino acid molecule, wherein said disease is characterised by increased immunological activity.

2. The method according to claim 1, wherein said amino acid molecule is mixed with a pharmaceutically compatible carrier.

3. The method according to claim 1, wherein said TIMP-1 analogue is a natural allelic variant of TIMP-1, and wherein said TIMP-1 analogue displays a homology of at least 70% relative to a TIMP-1 amino acid sequence (SEQ ID NO.: 9).

4. The method according to claim 1, wherein said TIMP-1 analogue is a recombinant allelic variant of TIMP-1, and wherein said TIMP-1 analogue displays a homology of at least 80% relative to a TIMP-1 amino acid sequence (SEQ ID NO.: 9).

5. The method according to claim 1, wherein said TIMP-1 analogue displays a homology of at least 95% relative to a TIMP-1 amino acid sequence (SEQ ID NO.: 9).

6. The method according to claim 1, wherein said TIMP-1 analogue has a length of at least 5 amino acid residues.

7. The method according to claim 1, wherein said amino acid molecule is operatively linked to a second amino acid sequence capable of modulating expression of said amino acid molecule.

8. The method according to claim 1, wherein said amino acid molecule is used for treatment of immune diseases, wherein said immune diseases are mediated by cells selected from a group consisting of Th1 cells, abnormally activated Th2 cells, activated CD8 or CD4 cells, activated eosinophilic granulocytes, mast cells, and abnormally secreting cells.

9. The method according to claim 8, wherein said abnormally secreting cells are epithelial cells of the nose and of the bronchial system.

10. A method according to claim 1, wherein said amino acid molecule is used to treatment a disease selected from a group consisting of multiple sclerosis, Crohn's disease, acute and chronic graft-versus-host diseases, acute transplant rejection, type 1 diabetes mellitus, rheumatoid arthritis, Lyme arthritis, reactive Yersinia-induced arthritis, post-streptoccocus cardiac valve and myocardial diseases, hepatitis C-induced chronic hepatitis, Hashimoto's thyroiditis, Grave's disease, primary sclerosing cholangitis, helicobacter pylori-induced gastrititis, cerebral malaria, contact dermatitis, aplastic anaemia, immunologically provoked abortions, bronchial asthma, sunburn, hay fever, and autoimmune disease.

11. The method according claim 1, wherein said amino acid molecule is present as a solution selected from the group consisting of injection solution, infusion solution, nose drops or nose sprays, drops, mouth wash, inhalants, tablets, plaster or cream.

12. A method for production of a medicament and the treatment of a disease that is characterised by an increased immunological activity comprising:
(a) producing a nucleic acid molecule having a nucleic acid sequence encoding a peptide selected from the group consisting of TIMP-1, a TIMP-1 analogue, and fragments of either that are capable of immunosuppressive activity;
(b) expressing said peptide from said nucleic acid molecule;
(c) contacting said peptide with a pharmaceutically compatible carrier; and
(c) treating a patient in need of immunosuppression with said medicament.

13. A compound for a rinsing solution for transplants, wherein said compound comprises a pharmaceutically compatible carrier and an amino acid molecule having an amino acid sequence encoding a protein selected from the group consisting of TIMP-1, a TIMP-1 analogue, and fragments of either that are capable of immunosuppressive activity.

14. A compound comprising an active ingredient that contains a TIMP-1 protein or fragment thereof, wherein said active ingredient is capable of having an immunosuppressive effect without inducing apoptosis in T-cells.

15. A method of making a pharmaceutical composition comprising:
(a) providing a nucleic acid molecule having a nucleic acid sequence encoding a protein selected from the group consisting of TIMP-1, a TIMP-1 analogue, and fragments of either that are capable of immunosuppressive activity;
(b) expressing said protein;
(c) preparing said pharmaceutical composition containing said protein.

16. The method according to claim 15, wherein said TIMP-1 analogue is a natural allelic variant of TIMP-1, and wherein said TIMP-1 analogue displays a homology of at least 70% relative to a TIMP-1 amino acid sequence (SEQ ID NO.: 9).

17. The method according to claim 15, wherein said TIMP-1 analogue is a recombinant allelic variant of TIMP-1, and wherein said TIMP-1 analogue displays a homology of at least 80% relative to a TIMP-1 amino acid sequence (SEQ ID NO.: 9).

18. The method according to claim 15, wherein said TIMP-1 analogue displays a homology of at least 95% relative to a TIMP-1 amino acid sequence (SEQ ID NO.: 9).

19. The method according to claim 15, wherein said TIMP-1 analogue has a length of at least 5 amino acids.

20. The method according to claim 15, wherein said nucleic acid sequence encoding a protein is operatively linked to a nucleic acid sequence capable of modulating expression of said nucleic acid sequence encoding a protein.

21. The method according to claim 15, wherein said pharmaceutical composition is used for treatment of immune diseases, wherein said immune diseases are mediated by cells selected from a group consisting of Th1 cells, abnormally activated Th2 cells, activated CD8 or CD4 cells, activated eosinophilic granulocytes, mast cells, and abnormally secreting cells.

22. The method according to claim 21, wherein said abnormally secreting cells are epithelial cells selected from the group consisting of epithelial cells of the nose and epithelial cells of the bronchial system.

23. A method according to claim 15, wherein said pharmaceutical composition is used to treatment a disease selected from a group consisting of multiple sclerosis, Crohn's disease, acute and chronic graft-versus-host diseases, acute transplant rejection, type 1 diabetes mellitus, rheumatoid arthritis, Lyme arthritis, reactive Yersinia-induced arthritis, post-streptoccocus cardiac valve and myocardial diseases, hepatitis C-induced chronic hepatitis, Hashimoto's thyroiditis, Grave's disease, primary sclerosing cholangitis, helicobacter pylori-induced gastrititis, cerebral malaria, contact dermatitis, aplastic anaemia, immunologically provoked abortions, bronchial asthma, sunburn, hay fever, and autoimmune disease.

24. The method according to claim 15, wherein said pharmaceutical composition is part of a solution selected from the group consisting of injection solution, infusion solution, nose drops or nose sprays, drops, mouth wash, inhalants, tablets, plaster or cream.

25. The method according to claim 15, wherein said pharmaceutical composition is used for the in vitro treatment of tissue before transplantation.

26. A method of treating a patient having a disease, wherein said disease is characterised by increased immunological activity, comprising:
(a) isolating a nucleic acid molecule having a nucleic acid sequence encoding a protein selected from the group consisting of TIMP-1, a TIMP-1 analogue, and fragments of either that are capable of immunosuppressive activity;
(b) preparing an expression construct suitable for transformation of target cells from said patient, said expression construct comprising said nucleic acid molecule; and
(c) treating said target cells from said patient having said disease, wherein said disease is characterised by increased immunological activity.

27. A compound for a rinsing solution for transplants, wherein said compound comprises a pharmaceutically compatible carrier and an nucleic acid sequence encoding a protein selected from the group consisting of TIMP-1, a TIMP-1 analogue, and fragments of either that are capable of immunosuppressive activity.

28. A method of T-cell purging comprising:
(a) preparing a rinsing solution for transplants, wherein said rinsing solution contains a protein selected from the group consisting of TIMP-1, a TIMP-1 analogue, and fragments of either that are capable of immunosuppressive activity;
(b) rinsing transplants in vitro with said rinsing solution.

29. The method of claim 28, further comprising (c) adding a pharmaceutically compatible carrier to said rinsing solution.


SEQUENCE LISTING (10 sequences)

SEQ ID NO: 1 - protein, 8 aa, Artificial
  Synthetic Construct
IMDQPFSV

SEQ ID NO: 2 - DNA, 21 bp, Artificial
  Synthetic Construct
ggacgagaaagagtgcctcaa

SEQ ID NO: 3 - DNA, 19 bp, Artificial
  Synthetic Construct
tgggacgactccagcttca

SEQ ID NO: 4 - DNA, 20 bp, Artificial
  Synthetic Construct
aggtgcccctgcccgacagc

SEQ ID NO: 5 - DNA, 19 bp, Artificial
  Synthetic Construct
atagcacccccaccactgg

SEQ ID NO: 6 - DNA, 20 bp, Artificial
  Synthetic Construct
ggcatttagggacagtggga

SEQ ID NO: 7 - DNA, 24 bp, Artificial
  Synthetic Construct
caggaggtctggcgggaacagagg

SEQ ID NO: 8 - DNA, 624 bp, Artificial
  Synthetic Construct
atggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagtttcgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctga

SEQ ID NO: 9 - protein, 207 aa, Artificial
  Synthetic construct
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 10 - protein, 220 aa, Artificial
  Synthetic construct
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP